(19) United States
(12) Patent Application Publication
(10) Pub. No.: US 2006/0073172 A1
(43) Pub. Date: Apr. 6, 2006

(54) Stabilized ophthalmic solution for the treatment of glaucoma and lowering intraocular pressure

(76) Inventors: L. Wayne Schneider, Crowley, TX (US); Wesley Wehsin Han, Arlington, TX (US); Bhagwati P. Kabra, Euless, TX (US); Owen Gan, Arlington, TX (US); John C. Liao, Fort Worth, TX (US); Huixiang Zhang, Fort Worth, TX (US)

Correspondence Address: Alcon Research, Ltd.;(Q-148), 6201 South Freeway, Fort Worth, TX, 76134-2099, US

(21) Appl. No.: 10/956,865

(22) Filed: Oct. 1, 2004

(51) Int. Cl. A61K 31/736; A61K 31/4162; A61K 31/403; A61K 9/00

(52) U.S. Cl. 424/400; 514/406; 514/411; 514/54

(57) ABSTRACT

The present invention provides stable ophthalmic solutions comprising a compound with serotonergic 5-HT₂ receptor activity and at least one stabilizer, together with methods of using such solutions to treat glaucoma and to lower intraocular pressure.

### BACKGROUND OF THE INVENTION

1. Field of the Invention

The present invention provides a stable ophthalmic solution comprising a compound with serotonergic 5-HT₂ receptor activity and at least one stabilizer.

2. Description of the Related Art

Serotonergic compounds which possess agonist activity at 5-HT₂ receptors have been found to effectively lower and control elevated IOP and may therefore be useful for treating glaucoma. Typically, the serotonergic compound is an indazole derivative containing both a hydroxyl group on the six member ring and an aminoalkyl side chain on the five member ring, such as 1-(2-aminopropyl)-indazol-6-ol (sometimes referred to hereinafter as “AL-34662”).

Generally, 5-HT₂ receptor-active serotonergic compounds are chemically not stable in aqueous solution at or near neutral pH. Structurally similar compounds such as epinephrine are usually formulated at an ophthalmic solution pH of about 4 to 5 in order to enhance their storage stability. Of course, for topical application to the eye, it is preferable to administer such compounds in aqueous solutions at or near the physiologic pH of 7.4, with a pH range of 6.0 to 8.0 generally being acceptable. Typically, oxidizable compounds such as 1-(2-aminopropyl)-indazol-6-ol (AL-34662) are stabilized by the addition of antioxidants. However, classical antioxidants, such as sodium metabisulfite (which is used to stabilize epinephrine), propyl gallate, and ascorbic acid did not stabilize 1-(2-aminopropyl)-indazol-6-ol.

### SUMMARY OF THE INVENTION

The known serotonergic compounds that may be useful for treating glaucoma are generally considered too unstable in solution to be useful in treatment. There is, therefore, a need for stable ophthalmic solutions containing serotonergic 5-HT₂ therapeutic agents suitable for topical administration to control IOP. The present invention provides a stable ophthalmic solution comprising a compound with serotonergic 5-HT₂ receptor activity and at least one stabilizer.

In preferred embodiments, the stabilizers present in the solution include one or more of the following: sodium thiosulfate pentahydrate (or any other salt or hydrate of thiosulfate), sodium iodide (or any other salt or hydrate of iodide), sodium sulfate (or any other salt or hydrate of sulfate), or xanthan gum.

Generally, the amount of serotonergic compound in the solution is from 0.01% to 5.0%, the amount of sodium thiosulfate pentahydrate is from 0.001% to 1.0%, the amount of sodium iodide is from 0.01% to 2.0%, the amount of sodium sulfate is from 0.01% to 3.0%, and the amount of xanthan gum is from 0.1% to 1.0%.

Preferably, the amount of serotonergic compound in the solution is from 0.06% to 2.0%, the amount of sodium thiosulfate pentahydrate is from 0.01% to 0.2%, the amount of sodium iodide is from 0.1% to 0.5%, the amount of sodium sulfate is from 0.5% to 1.5%, and the amount of xanthan gum is from 0.3% to 0.8%.

Most preferably, the amount of serotonergic compound in the solution is 1.0%, the amount of sodium thiosulfate pentahydrate is 0.05%, the amount of sodium iodide is 0.2%, the amount of sodium sulfate is 1.0%, and the amount of xanthan gum is 0.6%.

In other embodiments, the invention provides methods for treating glaucoma and/or lowering intraocular pressure (IOP) by administering a solution of the invention.

Typically, the solution of the invention will be administered topically.

### BRIEF DESCRIPTION OF THE DRAWINGS

The following drawings form part of the present specification and are included to further demonstrate certain aspects of the present invention. The invention may be better understood by reference to these drawings in combination with the detailed description of specific embodiments presented herein.

FIG. 1. Illustrates the effect of sodium thiosulfate pentahydrate on AL-34662 assay (percent of initial after correction for weight loss) in formulations with xanthan gum and sodium chloride at 50° C.

FIG. 2. Illustrates the effect of sodium thiosulfate pentahydrate on AL-34662 assay (percent of initial after correction for weight loss) in formulations with xanthan gum and sodium sulfate at 50° C.

FIG. 3. Illustrates the effect of sodium thiosulfate pentahydrate on AL-34662 assay (percent of initial after correction for weight loss) in formulations with hydroxypropyl methylcellulose (HPMC) at 50° C.

FIG. 4. Illustrates the effect of xanthan gum versus hydroxypropyl methylcellulose (HPMC) on AL-34662 assay (percent of initial after correction for weight loss) in formulations without sodium thiosulfate at 50° C.

FIG. 5. Illustrates effect of xanthan gum versus hydroxypropyl methylcellulose (HPMC) on AL-34662 assay (percent of initial after correction for weight loss) in formulations with sodium thiosulfate at 50° C.

FIG. 6. Illustrates the effect of sodium iodide, sodium sulfate and sodium chloride on AL-34662 assay (percent of initial after correction for weight loss) in formulations at 50° C.

### DETAILED DESCRIPTION OF PREFERRED EMBODIMENTS

Serotonergic compounds which possess agonist activity at 5-HT2 receptors have been found to effectively lower and control elevated IOP and are useful for treating glaucoma. However, such serotonergic compounds have typically been found to be unstable in solution, particularly in a solution for topical delivery to the eye. The present invention, for the first time, provides a stable ophthalmic solution containing serotonergic compounds having 5-HT₂ receptor activity that are useful for topical delivery to the eye for lowering and controlling intraocular pressure and treating glaucoma.

Specific compounds useful in the solutions of the present invention include: 1) 1-(2-aminopropyl)-indazol-6-ol (AL-34662); 2) (R)₄-iodo-2,5 dimethoxy-α-methyl-benzeneethanamine [(R)-DOI], the prototypical selective 5-HT2 agonist which is not selective amongst the 5-HT2 receptor subtypes (Baxter et al. 1995); 3) α-methyl-serotonin, a potent 5-HT2 agonist with modest receptor subtype selectivity: 5-HT2B>5-HT2C>5-HT2A (Baxter et al. 1995); 4) 5-methoxy-α-methyltryptamine, with a profile similar to that of α-methyl-serotonin (Nichols et al. 1998); 5) 1-((S)-2-aminopropyl)-8,9-dihydropyrano[3,2-e]indole; and 6) (R)-1-((S)-2-aminopropyl)-1,7,8,9-tetrahydro-pyrano[2,3-g]indazol-8-ol.

The following references are not limiting, but rather exemplify compounds useful according to the present invention and are incorporated herein by reference: U.S. Pat. Nos. 5,861,425; 5,646,173; 5,578,612; 5,571,833; 5,545,644; 5,494,928; 4,659,706 and 4,487,773; published European Patent Specification No. 863,136; published International Patent Application Nos. WO98/56768; WO98/31354; WO98/30548; WO98/30546; WO 01/70702. Additionally, compounds disclosed in the following publications further exemplify compounds useful according to the present invention and are also incorporated herein by reference: Parker et al. (1998); Vangveravong et al. (1998); Albertini et al., (1998); Monte et al. (1997); Bos et al. (1997a); Bos et al. (1997b); Monte et al. (1996); Glennon et al. (1994); Macor et al. (1994); Macor et al. (1992a); Macor et al. (1992b); Glennon et al. (1992); Seggel et al. (1990).

It is recognized that many of the aforementioned compounds have asymmetric atoms, therefore all enantiomers and diastereomers are contemplated. Also contemplated are pharmaceutically acceptable salts as well as the free bases of the compounds.

Generally, 5-HT₂ receptor-active serotonergic compounds are chemically unstable in aqueous solution at or near neutral pH. Structurally similar compounds such as epinephrine are usually formulated at an ophthalmic solution pH of about 4 to 5 in order to enhance their storage stability. Of course, for topical application to the eye, it is preferable to administer such compounds in aqueous solutions at or near the physiologic pH of 7.4, with a pH range of 6.0 to 8.0 generally being acceptable.

Clearly, what is needed for these serotonergic compounds is a delivery system that provides stability to the serotonergic compound and that is also safe for administration to the eye. The present invention provides, for the first time, a stable ophthalmic solution containing from 0.01% to 5% of a 5-HT₂ receptor-active serotonergic compound in the presence of at least one stabilizer.

Typically, oxidizable compounds such as 1-(2-aminopropyl)-indazol-6-ol (AL-34662) are stabilized by the addition of antioxidants. However, classical antioxidants, such as sodium metabisulfite (which is used for epinephrine), propyl gallate, and ascorbic acid did not stabilize 1-(2-aminopropyl)-indazol-6-ol. It has been shown that an aqueous solution of 1-(2-aminopropyl)-indazol-6-ol can be stabilized with the addition of one or more stabilizers.

In preferred embodiments, the stabilizers present in the solution include one or more of the following: sodium thiosulfate pentahydrate (or any other salt or hydrate of thiosulfate), sodium iodide (or any other salt or hydrate of iodide), sodium sulfate (or any other salt or hydrate of sulfate), or xanthan gum.

Generally, the amount of serotonergic compound in the solution is from 0.01% to 5.0%, the amount of sodium thiosulfate pentahydrate is from 0.01% to 1.0%, the amount of sodium iodide is from 0.01% to 2.0%, the amount of sodium sulfate is from 0.001% to 3.0%, and the amount of xanthan gum is from 0.1% to 1.0%. Unless otherwise indicated, all amounts reflected as percentages are understood to be weight percentages.

Preferably, the amount of serotonergic compound in the solution is from 0.01% to 2.0%, the amount of sodium thiosulfate pentahydrate is from 0.02% to 0.2%, the amount of sodium iodide is from 0.1% to 0.5%, the amount of sodium sulfate is from 0.5% to 1.5%, and the amount of xanthan gum is from 0.3% to 0.8%.

Most preferably, the amount of serotonergic compound in the solution is 1.0%, the amount of sodium thiosulfate pentahydrate is 0.02%, the amount of sodium iodide is 0.2%, the amount of sodium sulfate is 1.0%, and the amount of xanthan gum is 0.6%.

Sodium thiosulfate is an antioxidant that has been used in compositions of pranoprofen α-methyl-5H-[1]benzopyrano[2,3-b]pyridine-7-acetic acid), an anti-inflammatory agent (See U.S. Pat. Nos. 5,856,345 and 5,889,030); ortophen (diclofenac-sodium), for the treatment of eye inflammatory diseases (See RU Patent No. 2149611); 15-trans prostaglandin F2α (WO 91/14428); carbonic anhydrase inhibitors for elevated IOP (U.S. Pat. No. 4,438,123); compositions to care for contact lenses (U.S. Pat. No. 5,424,078; U.S. Pat. No. 5,387,394; U.S. Pat. No. 5,277,901); biodegradable ocular implants (U.S. Pat. Nos. 5,164,188; 4,997,652; 4,853,224); ophthalmic drug delivery system utilizing thermosetting gels (U.S. Pat. No. 4,474,751); celiprolol for glaucoma (U.S. Pat. No. 4,470,965); hepatocyte and keratinocyte growth factors for stimulating the proliferative and motility of corneal cells (U.S. Pat. Nos. 5,703,047; 5,589,451); diflupredonate, an anti-inflammatory and anti-allergic agent (U.S. Pat. No. 5,556,848); non-steroidal cyclooxygenase inhibitor for elevated IOP (U.S. Pat. Nos. 5,474,985; 5,486,540; 5,545,665; 5,587,391); cyclopentane (ene) heptenoic or heptanoic acid for ocular hypertension (U.S. Pat. Nos. 5,990,138; 5,906,989; 5,798,378; 5,681,848); tear stimulant (U.S. Pat. Nos. 5,961,987; 4,820,737); carbonic anhydrase inhibitors to increase retinal and optical nerve head blood flow (U.S. Pat. No. 5,789,435); oxazolinone (U.S. Pat. No. 6,551,584); epinastin (U.S. Published Application No. 2003050303); quinolone carboxylic acid (U.S. Published Application No. 2002187193); azalide antibiotic compositions (U.S. Published Application No. 2002019353).

Prior to their use by the inventors, sodium thiosulfate, sodium iodide, sodium sulfate, and xanthan gum have not been used to stabilize formulations including serotonergic compounds in ophthalmic solutions. Their stabilizing effects on serotonergic compounds, a new class of compound, was unexpected in light of the inability of classical antioxidants, such as sodium metabisulfite (used for Epinephrine), propyl gallate, and ascorbic acid to stabilize 1-(2-aminopropyl)-indazol-6-ol. Therefore, their ability to adequately stabilize the serotonergic compounds, as shown for the first time by the present inventors, was surprising.

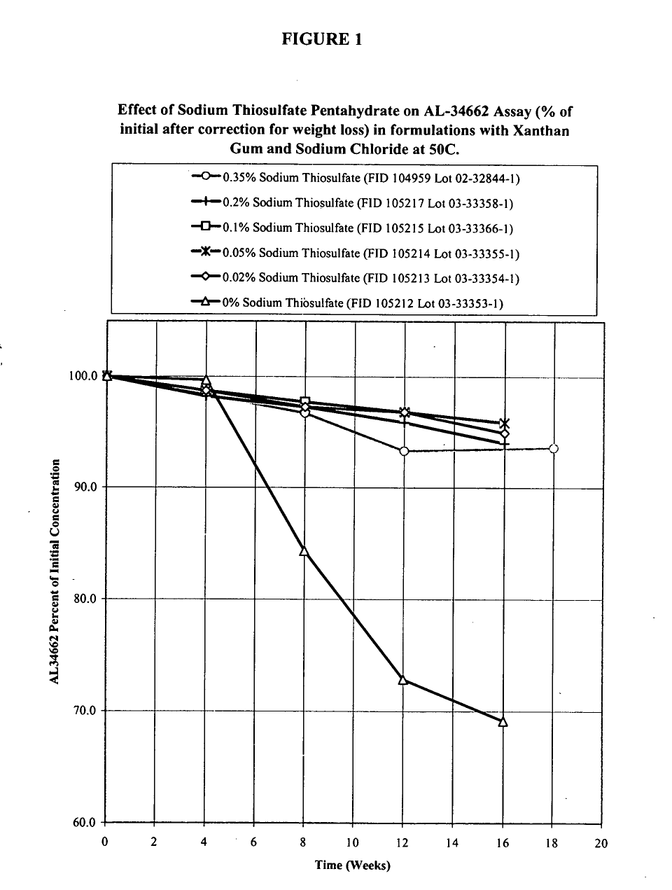

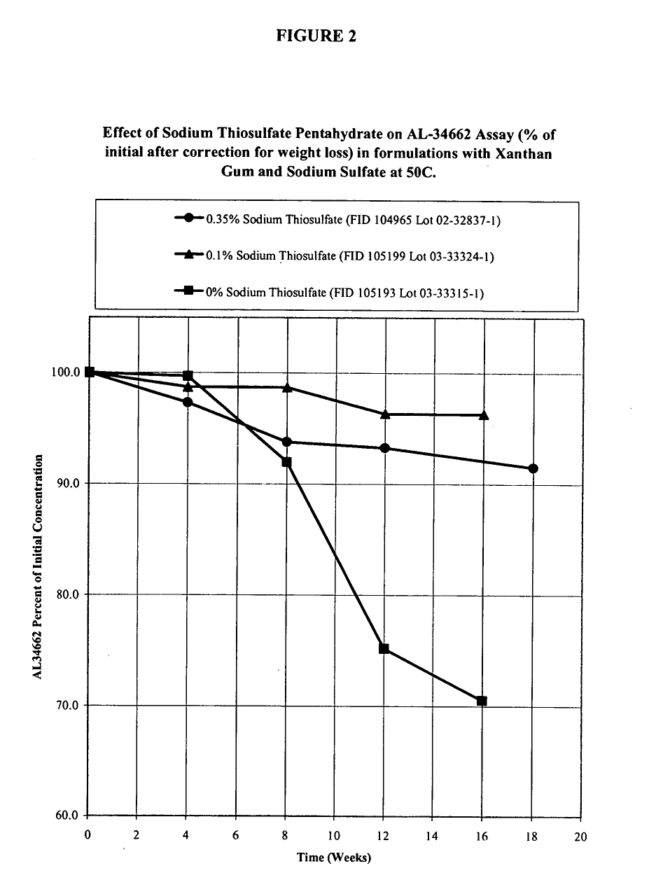

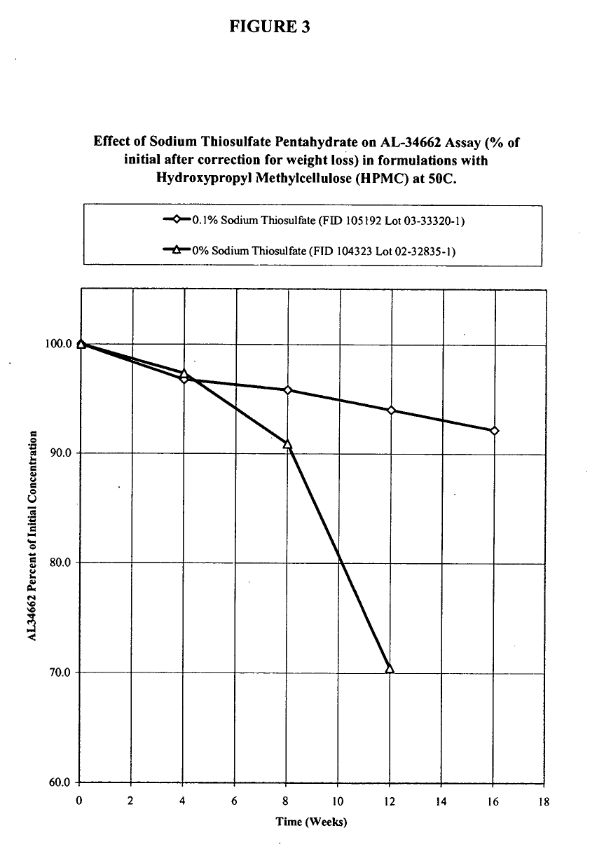

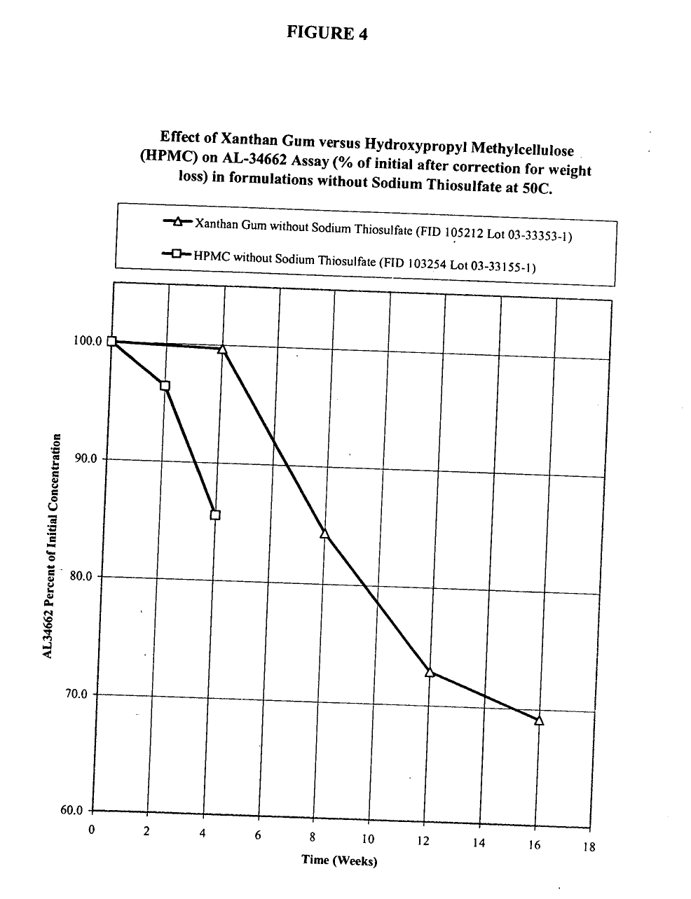

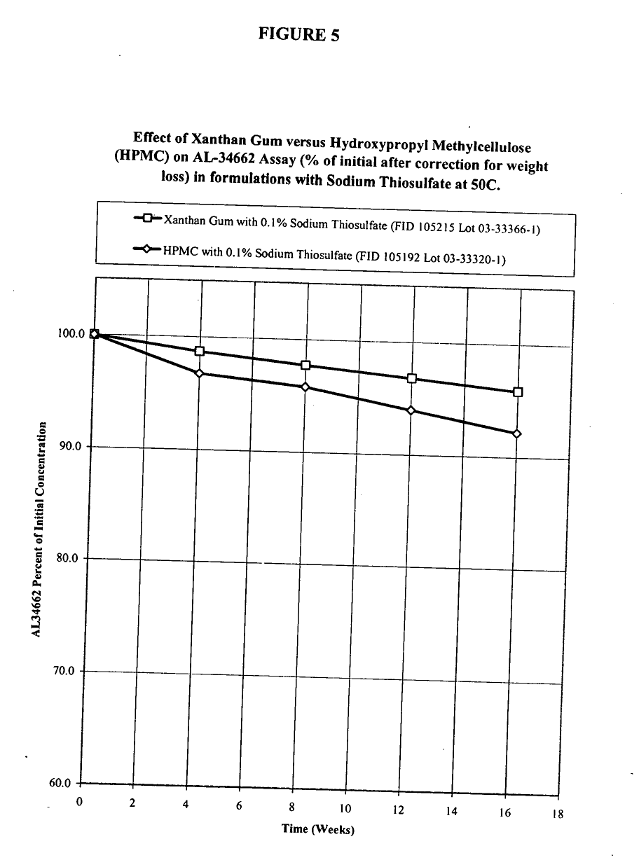

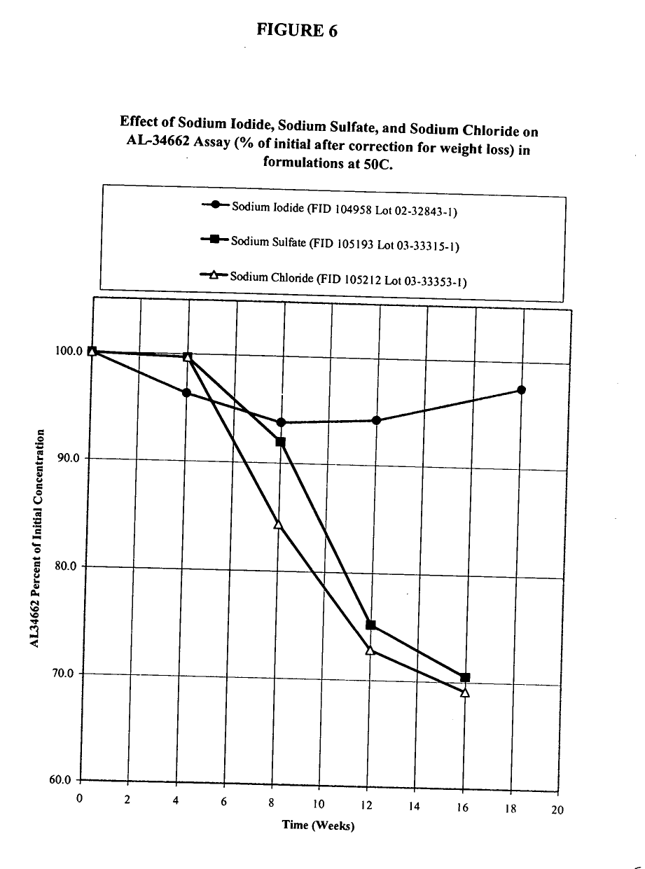

When a stabilizer is added to an aqueous solution of 1-(2-aminopropyl)-indazol-6-ol, the stability of the solution has been shown to increase over the same solution or a similar solution of the compound not including the stabilizer. For example, the stability of a 1.0% aqueous solution of 1-(2-aminopropyl)-indazol-6-ol in the presence of various concentrations of sodium thiosulfate is shown in Examples 1, 2, and 3, and FIGS. 1, 2, and 3. The stability of a 1.0% aqueous solution of 1-(2-aminopropyl)-indazol-6-ol in the presence of xanthan gum versus hydroxypropyl methylcellulose (HPMC) is shown in Examples 4 and 5, and FIGS. 4 and 5. The stability of a 1.0% aqueous solution of 1-(2-aminopropyl)-indazol-6-ol in the presence of sodium iodide or sodium sulfate versus sodium chloride is shown in Example 6 and FIG. 6.

The compounds are administered to the eye (e.g., topically, intracamerally, or via an implant). The compounds and stabilizers are preferably incorporated into topical ophthalmic formulations for delivery to the eye. The compounds and stabilizers may be combined with ophthalmologically acceptable preservatives, surfactants, viscosity enhancers, penetration enhancers, tonicity reagents, and water to form an aqueous, sterile ophthalmic solution or suspension. Ophthalmic solution formulations may be prepared by dissolving a compound and stabilizer in a physiologically acceptable isotonic aqueous buffer. In order to prepare sterile ophthalmic ointment formulations, the compound is combined with a stabilizer in an appropriate ointment vehicle, such as, mineral oil, liquid lanolin, or white petrolatum.

The compounds and stabilizers are preferably formulated as topical ophthalmic solutions or suspensions, with a pH of about 6 to 8. The compounds will normally be contained in these formulations in an amount 0.01% to 5% by weight, but preferably in an amount of 0.2% to 1% by weight. Thus, for topical presentation 1 to 2 drops of these formulations would be delivered to the surface of the eye 1 to 4 times per day according to the routine discretion of a skilled clinician.

The compounds and stabilizers can also be used in combination with other agents for treating glaucoma, such as, but not limited to, β-blockers, prostaglandin analogues, carbonic anhydrase inhibitors, α2 agonists and miotics. The compounds and stabilizers can also be used with calcium channel blockers and antagonists for metabotropic and ionotropic glutamate receptors and/or antagonists for their associated binding sites, such as, the polyamine and strychnine-insensitive glycine sites. These agents may be administered topically, but usually systemically.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Effect of Sodium Thiosulfate Pentahydrate Concentration on AL-34662 Stability in Formulations with Xanthan Gum and Sodium Chloride

Formulations of 1% AL-34662 with xanthan gum, sodium chloride, and different concentrations of sodium thiosulfate pentahydrate are listed in Table 1A. These formulations were prepared using the compounding procedure described below. The formulations were packaged in standard 3 mL or 5 mL polyethylene DROP-TAINER® bottles and their stability was studied at 50° C. The results of the active compound (AL-34662) assay corrected for weight loss (which is usual for an aqueous product packaged in a semi-permeable container) are provided in Table 1B. The time it takes for the AL-34662 assay to drop below 95% of initial and 90% of initial is given in Table 1C. The results in Table 1C show that the presence of sodium thiosulfate pentahydrate prolongs the stability of AL-34662. The effect of prolonging the stability of AL-34662 formulations is maximal at sodium thiosulfate pentahydrate concentrations of 0.05% to 0.10%. The effect of prolonging the stability of AL-34662 is slightly less at the higher sodium thiosulfate pentahydrate concentration of 0.35%.

Typical Serotonergic Ophthalmic Formulation Compounding Procedure

A. Polymer Stock Solution (Xanthan Gum, Hydroxypropyl Methylcellulose, or Other Polymer)

Uniformly disperse and hydrate the appropriate quantity of polymer in the appropriate quantity of purified water. Polish the polymer stock solution by filtration with an appropriate size filter (e.g., 0.4 to 20 microns). Sterilize the polymer stock solution by autoclaving with a cycle equivalent to at least 30 minutes at 121° C. Cool to room temperature and mix until homogeneous.

B. Concentrated Solution of Remaining Ingredients

1. In a suitable mixing vessel, weigh and dissolve the batch quantities of the remaining ingredients with a fixed concentration except the active ingredient (e.g., AL-34662) and the preservative (e.g., benzododecinium bromide or benzalkonium chloride) in a suitable quantity of purified water at room temperature.
2. Weigh and transfer the batch quantity of active ingredient (e.g., AL-34662) to the vessel with a suitable quantity of purified water.
3. While mixing, add an appropriate quantity of acid (e.g., hydrochloric or sulfuric acid) or base (e.g., sodium hydroxide) and continue stirring until the active ingredient has dissolved. When the active ingredient is completely dissolved, measure and adjust the pH of the solution (e.g., 7.5±0.1) with acid and/or base.
4. Weigh and add the batch quantity of preservative (e.g., benzododecinium bromide or benzalkonium chloride) and add purified water to about 40% of the batch quantity. Mix until homogeneous.
   C. Final Compounding and Filling*

Weigh the appropriate quantity of sterile polymer stock solution from A. above into a suitable sterile vessel. Filter the concentrated solution from B. above with an appropriate sterile filter (e.g., 0.2 microns) and add to the sterile polymer stock solution. Rinse the sterile filter with purified water and add the filtered rinse to the sterile vessel. Adjust to 100% of the batch quantity with sterile filtered purified water and mix until homogeneous. Fill into presterilized 3 mL or 5 mL polyethylene DROP-TAINER® bottles. Plug, cap, and label the bottles.

*Note: To impart sterility to the final formulation, this step must be carried out in a clean suite, laminar flow hood, or other sterile environment.

TABLE 1A
Composition of formulations with Sodium Thiosulfate Pentahydrate, Xanthan
Gum, and Sodium Chloride.

| | Formulation ID Number (FID) | | | | | |
|---|---|---|---|---|---|---|
| | 105212 | 105213 | 105214 | 105215 | 105217 | 104959 |
| | Lot Number | | | | | |
| | 03-33353-1 | 03-33354-1 | 03-33355-1 | 03-33366-1 | 03-33358-1 | 02-32844-1 |
| COMPONENT | % w/v | % w/v | % w/v | % w/v | % w/v | % w/v |
| AL-34662 | 1 | 1 | 1 | 1 | 1 | 1 |
| Sodium Thiosulfate | 0 | 0.02 | 0.05 | 0.10 | 0.20 | 0.35 |
| Pentahydrate | | | | | | |
| Xanthan Gum | 0.6 | 0.6 | 0.6 | 0.6 | 0.6 | 0.6 |
| Sodium Chloride | 0.5 | 0.49 | 0.48 | 0.47 | 0.44 | 0.4 |
| Polysorbate 80 | 0.05 | 0.05 | 0.05 | 0.05 | 0.05 | 0.05 |
| Monosodium | 0.23 | 0.23 | 0.23 | 0.23 | 0.23 | 0.23 |
| Phosphate Dihydrate | | | | | | |
| Disodium Edetate | 0.01 | 0.01 | 0.01 | 0.01 | 0.01 | 0.01 |
| Dihydrate | | | | | | |
| Benzododecinium | 0.012 | 0.012 | 0.012 | 0.012 | 0.012 | 0.012 |
| Bromide | | | | | | |
| Sodium Hydroxide | 7.5 ± 0.1 | 7.5 ± 0.1 | 7.5 ± 0.1 | 7.5 ± 0.1 | 7.5 ± 0.1 | 7.5 ± 0.1 |
| q.s. pH | | | | | | |
| Hydrochloric Acid | 7.5 ± 0.1 | 7.5 ± 0.1 | 7.5 ± 0.1 | 7.5 ± 0.1 | 7.5 ± 0.1 | 7.5 ± 0.1 |
| q.s. pH | | | | | | |
| Purified Water q.s. % | 100 | 100 | 100 | 100 | 100 | 100 |

TABLE 1B
Effect of Sodium Thiosulfate Pentahydrate on AL-34662 Assay (% of initial
after correction for weight loss) in formulations with Xanthan Gum and
Sodium Chloride at 50° C.

| | FID | | | | | |
|---|---|---|---|---|---|---|
| | 105212 | 105213 | 105214 | 105215 | 105217 | 104959 |
| | Lot Number | | | | | |
| | 03-33353-1 | 03-33354-1 | 03-33355-1 | 03-33366-1 | 03-33358-1 | 02-32844-1 |
| | Critical Component | | | | | |
| | 0% | 0.02% | 0.05% | 0.10% | 0.20% | 0.35% |
| | Sodium | Sodium | Sodium | Sodium | Sodium | Sodium |
| Age in | Thiosulfate | Thiosulfate | Thiosulfate | Thiosulfate | Thiosulfate | Thiosulfate |
| Weeks | % Initial | % Initial | % Initial | % Initial | % Initial | % Initial |
| 0 | 100 | 100 | 100 | 100 | 100 | 100 |
| 4 | 100 | 99 | 99 | 99 | 98 | 98 |
| 8 | 84 | 97 | 97 | 98 | 97 | 97 |
| 12 | 73 | 97 | 97 | 97 | 96 | 93 |
| 16 | 69 | 95 | 96 | 96 | 94 | ND |
| 18 | ND | ND | ND | ND | ND | 94 |

ND = Not Determined

TABLE 1C
Effect of Sodium Thiosulfate Pentahydrate on the time it takes for the AL-
34662 Assay to drop below 95% and 90% of initial value in formulations with Xanthan
Gum and Sodium Chloride at 50° C.

| | FID | | | | | |
|---|---|---|---|---|---|---|
| | 105212 | 105213 | 105214 | 105215 | 105217 | 104959 |
| | Lot Number | | | | | |
| | 03-33353-1 | 03-33354-1 | 03-33355-1 | 03-33366-1 | 03-33358-1 | 02-32844-1 |
| | Critical Component | | | | | |
| | 0% | 0.02% | 0.05% | 0.10% | 0.20% | 0.35% |
| | Sodium | Sodium | Sodium | Sodium | Sodium | Sodium |
| | Thiosulfate | Thiosulfate | Thiosulfate | Thiosulfate | Thiosulfate | Thiosulfate |
| Age in weeks | Between 4 | More than 16 | More than 16 | More than 16 | Between 12 | Between 8 |
| for AL-34662 | and 8 weeks | weeks | weeks | weeks | and 16 weeks | and 12 |
| assay to drop | | | | | | weeks |
| below 95% | | | | | | |
| label | | | | | | |
| Age in | Between 4 | More than | More than | More than | More than | More than |
| weeks for | and 8 | 16 weeks | 16 weeks | 16 weeks | 16 weeks | 18 weeks |
| AL-34662 | weeks | | | | | |
| assay to | | | | | | |
| drop below | | | | | | |
| 90% label | | | | | | |

### EXAMPLE 2

### Effect of Sodium Thiosulfate Pentahydrate Concentration on AL-34662 Stability in Formulations with Xanthan Gum and Sodium Sulfate

Formulations of 1% AL-34662 with xanthan gum, sodium sulfate, and different concentrations of sodium thiosulfate pentahydrate are listed in Table 2A. These formulations were prepared using the procedure described in Example 1. These formulations were packaged in standard 3 mL or 5 mL polyethylene DROP-TAINER® bottles and their stability was studied at 50° C. The results of the active compound (AL-34662) assay corrected for weight loss (which is usual for an aqueous product packaged in a semi-permeable container) are provided in Table 2B. The time it takes for the AL-34662 assay to drop below 95% of initial and 90% of initial is given in Table 2C. The results in Table 2C show that the presence of sodium thiosulfate pentahydrate prolongs the stability of AL-34662. The effect of prolonging the stability of AL-34662 with sodium thiosulfate pentahydrate is better at 0.10% sodium thiosulfate pentahydrate concentration than at 0.35% sodium thiosulfate pentahydrate concentration.

TABLE 2A
Composition of formulations with Sodium Thiosulfate Pentahydrate,
Xanthan Gum, and Sodium Sulfate.

| | Formulation ID Number (FID) | | |
|---|---|---|---|
| | 105193 | 105199 | 104965 |
| | Lot Number | | |
| | 03-33315-1 | 03-33324-1 | 02-32837-1 |
| COMPONENT | % w/v | % w/v | % w/v |
| AL-34662 | 1 | 1 | 1 |
| Sodium Thiosulfate | 0 | 0.10 | 0.35 |
| Pentahydrate | | | |
| Xanthan Gum | 0.6 | 0.6 | 0.6 |
| Sodium Sulfate | 1.05 | 1.00 | 0.84 |
| Polysorbate 80 | 0.05 | 0.05 | 0.05 |
| Monosodium Phosphate | 0.23 | 0.23 | 0.23 |
| Dihydrate | | | |
| Disodium Edetate Dihydrate | 0.01 | 0.01 | 0.01 |
| Benzododecinium Bromide | 0.012 | 0.012 | 0.012 |
| Sodium Hydroxide q.s. pH | 7.5 ± 0.1 | 7.5 ± 0.1 | 7.5 ± 0.1 |
| Hydrochloric Acid q.s. pH | 7.5 ± 0.1 | 7.5 ± 0.1 | — |
| Sulfuric Acid q.s. pH | — | — | 7.5 ± 0.1 |
| Purified Water q.s. % | 100 | 100 | 100 |

TABLE 2B
Effect of Sodium Thiosulfate Pentahydrate on AL-34662 Assay
(% of initial after correction for weight loss) in formulations with
Xanthan Gum and Sodium Sulfate at 50° C.

| | FID | | |
|---|---|---|---|
| | 105193 | 105199 | 104965 |
| | Lot Number | | |
| | 03-33315-1 | 03-33324-1 | 02-32837-1 |
| | Critical Component | | |
| | 0% | 0.1% | 0.35% |
| | Sodium | Sodium | Sodium |
| | Thiosulfate | Thiosulfate | Thiosulfate |
| Age in Weeks | % Initial | % Initial | % Initial |
| 0 | 100 | 100 | 100 |
| 4 | 100 | 99 | 97 |
| 8 | 92 | 99 | 94 |
| 12 | 75 | 96 | 93 |
| 16 | 71 | 96 | ND |
| 18 | ND | ND | 92 |

ND = Not Determined

TABLE 2C
Effect of Sodium Thiosulfate Pentahydrate on the time it takes for the
AL-34662 Assay to drop below 95% and 90% of initial value in
formulations with Xanthan Gum and Sodium Sulfate at 50° C.

| | FID | | |
|---|---|---|---|
| | 105193 | 105199 | 104965 |
| | Lot Number | | |
| | 03-33315-1 | 03-33324-1 | 02-32837-1 |
| | Critical Component | | |
| | 0% | 0.1% | 0.35% |
| | Sodium | Sodium | Sodium |
| | Thiosulfate | Thiosulfate | Thiosulfate |
| Age in weeks for AL- | Between 4 and | More than 16 | Between 4 and |
| 34662 assay to drop | 8 weeks | weeks | 8 weeks |
| below 95% label | | | |
| Age in weeks for AL- | Between 8 and | More than 16 | More than 18 |
| 34662 assay to drop | 12 weeks | weeks | weeks |
| below 90% label | | | |

### EXAMPLE 3

### Effect of Sodium Thiosulfate Pentahydrate on AL-34662 stability in Formulations with Hydroxypropyl Methylcellulose (HPMC)

Formulations of 1% AL-34662 with hydroxypropyl methylcellulose (HPMC) and different concentrations of sodium thiosulfate pentahydrate are listed in Table 3A. These formulations were prepared using the procedure described in Example 1. These formulations were packaged in standard 3 mL or 5 mL polyethylene DROP-TAINER® bottles and their stability was studied at 50° C. The results of the active compound (AL-34662) assay corrected for weight loss (which is usual for an aqueous product packaged in a semi-permeable container) are provided in Table 3B. The time it takes for the AL-34662 assay to drop below 95% of initial and 90% of initial is given in Table 3C. The results in Table 3C show that sodium thiosulfate pentahydrate prolongs the stability of AL-34662 in the presence of hydroxypropyl methylcellulose (HPMC).

TABLE 3A
Composition of formulations with Sodium Thiosulfate Pentahydrate
and Hydroxypropyl Methylcellulose (HPMC).

| | Formulation ID Number (FID) | |
|---|---|---|
| | 104323 | 105192 |
| | Lot Number | |
| | 02-32835-1 | 03-33320-1 |
| COMPONENT | % w/v | % w/v |
| AL-34662 | 1 | 1 |
| Sodium Thiosulfate Pentahydrate | 0 | 0.10 |
| Hydroxypropyl Methylcellulose (HPMC) | 0.88 | 0.88 |
| Sodium Chloride | 0.55 | 0.52 |
| Polysorbate 80 | 0.05 | 0.05 |
| Monosodium Phosphate Dihydrate | 0.23 | 0.23 |
| Benzalkonium Chloride | 0.01 | 0.01 |
| Sodium Hydroxide q.s. pH | 7.5 ± 0.1 | 7.5 ± 0.1 |
| Hydrochloric Acid q.s. pH | 7.5 ± 0.1 | 7.5 ± 0.1 |
| Purified Water q.s. % | 100 | 100 |

TABLE 3B
Effect of Sodium Thiosulfate Pentahydrate on AL-34662 Assay
(% of initial after correction for weight loss) in formulations with
Hydroxypropyl Methylcellulose (HPMC) at 50° C.

FID

| | |
|---|---|
| 104323 | 105192 |

Lot Number

| | |
|---|---|
| 02-32835-1 | 03-33320-1 |

Critical Component

| | 0% | 0.1% |
|---|---|---|
| | Sodium | Sodium |
| | Thiosulfate | Thiosulfate |
| Age in Weeks | % Initial | % Initial |
| 0 | 100 | 100 |
| 4 | 97 | 97 |
| 8 | 91 | 96 |
| 12 | 71 | 94 |
| 16 | ND | 92 |

ND = Not Determined

TABLE 3C
Effect of Sodium Thiosulfate Pentahydrate on the time it takes for the
AL-34662 Assay to drop below 95% and 90% of initial value in
formulations with Hydroxypropyl Methylcellulose (HPMC) at 50° C.

FID

| | |
|---|---|
| 104323 | 105192 |

Lot Number

| | |
|---|---|
| 02-32835-1 | 03-33320-1 |

Critical Component

| | 0% Sodium | 0.10% Sodium |
|---|---|---|
| | Thiosulfate | Thiosulfate |
| Age in weeks for AL- | Between 4 and | Between 8 and |
| 34662 assay to drop | 8 weeks | 12 weeks |
| below 95% label | | |
| Age in weeks for AL- | Between 8 and | More than 16 |
| 34662 assay to drop | 12 weeks | weeks |
| below 90% label | | |

### EXAMPLE 4

### Effect of Xanthan Gum Versus Hydroxypropyl Methylcellulose (HPMC) on AL-34662 Stability in Formulations without Sodium Thiosulfate

Formulations of 1% AL-34662 with xanthan gum or hydroxypropyl methylcellulose (HPMC) without sodium thiosulfate are listed in Table 4A. These formulations were prepared using the procedure described in Example 1. These formulations were packaged in standard or 5 mL polyethylene DROP-TAINER® bottles and their stability was studied at 50° C. The results of the active compound (AL-34662) assay corrected for weight loss (which is usual for an aqueous product packaged in a semi-permeable container) are provided in Table 4B. The time it takes for the AL-34662 assay to drop below 95% of initial and 90% of initial is given in Table 4C. The results in Table 4C show that xanthan gum prolongs the stability of the AL-34662 (i.e., the AL-34662 assay levels are above 90% of initial for a longer period of time) compared to hydroxypropyl methylcellulose (HPMC).

TABLE 4A
Composition of formulations with either Xanthan Gum or
Hydroxypropyl Methylcellulose (HPMC), without Sodium Thiosulfate.

| | Formulation ID Number (FID) | |
|---|---|---|
| | 105212 | 103254 |
| | Lot Number | |
| | 03-33353-1 | 03-33155-1 |
| COMPONENT | % w/v | % w/v |
| AL-34662 | 1 | 1 |
| Xanthan Gum | 0.6 | — |
| Hydroxypropyl Methylcellulose (HPMC) | — | 0.8 |
| Sodium Chloride | 0.5 | 0.55 |
| Polysorbate 80 | 0.05 | 0.05 |
| Monosodium Phosphate Dihydrate | 0.23 | — |
| Monosodium Phosphate Monohydrate | — | 0.2 |
| Disodium Edetate Dihydrate | 0.01 | 0.01 |
| Benzododecinium Bromide | 0.012 | — |
| Benzalkonium Chloride | — | 0.01 |
| Sodium Hydroxide q.s. pH | 7.5 ± 0.1 | 7.5 ± 0.1 |
| Hydrochloric Acid q.s. pH | 7.5 ± 0.1 | 7.5 ± 0.1 |
| Purified Water q.s. % | 100 | 100 |

TABLE 4B
Effect of Xanthan Gum versus Hydroxypropyl Methylcellulose
(HPMC) on AL-34662 Assay (% of initial after correction for
weight loss) in formulations without Sodium Thiosulfate at 50° C.

FID

| | |
|---|---|
| 105212 | 103254 |

Lot Number

| | |
|---|---|
| 03-33353-1 | 03-33155-1 |

Critical Component

| | Xanthan Gum | HPMC |
|---|---|---|
| Age in Weeks | % Initial | % Initial |
| 0 | 100 | 100 |
| 2 | ND | 96 |
| 4 | 100 | 86 |
| 8 | 84 | ND |
| 12 | 73 | ND |
| 16 | 69 | ND |

ND = Not Determined

TABLE 4C
Effect of Xanthan Gum versus Hydroxypropyl Methylcellulose
(HPMC) on the time it takes for the AL-34662 Assay to drop
below 95% and 90% of initial value in formulations without
Sodium Thiosulfate at 50° C.

FID

| | |
|---|---|
| 105212 | 103254 |

Lot Number

| | |
|---|---|
| 03-33353-1 | 03-33155-1 |

Critical Component

| | Xanthan Gum | HPMC |
|---|---|---|
| Age in weeks for AL-34662 | Between 4 and | Between 2 and |
| assay to drop | 8 weeks | 4 weeks |
| below 95% label | | |
| Age in weeks for AL-34662 | Between 4 and | Between 2 and |
| assay to drop | 8 weeks | 4 weeks |
| below 90% label | | |

### EXAMPLE 5

### Effect of Xanthan Gum Versus Hydroxypropyl Methylcellulose (HPMC) on AL-34662 Stability in Formulations with Sodium Thiosulfate

Formulations of 1% AL-34662 with xanthan gum or hydroxypropyl methylcellulose (HPMC) with sodium thiosulfate are listed in Table 5A. These formulations were prepared using the procedure described in Example 1. These formulations were packaged in standard or 5 mL polyethylene DROP-TAINER® bottles and their stability was studied at 50° C. The results of the active compound (AL-34662) assay corrected for weight loss (which is usual for an aqueous product packaged in a semi-permeable container) are provided in Table 5B. The time it takes for the AL-34662 assay to drop below 95% of initial and 90% of initial is given in Table 5C. The results in Table 5C show that in the presence of sodium thiosulfate, xanthan gum prolongs the stability of AL-34662 compared to that of hydroxypropyl methylcellulose (HPMC).

TABLE 5A
Composition of formulations with either Xanthan Gum or
Hydroxypropyl Methylcellulose (HPMC), with Sodium Thiosulfate.

| | Formulation ID Number (FID) | |
|---|---|---|
| | 105215 | 105192 |
| | Lot Number | |
| | 03-33366-1 | 03-33320-1 |
| COMPONENT | % w/v | % w/v |
| AL-34662 | 1 | 1 |
| Sodium Thiosulfate Pentahydrate | 0.1 | 0.1 |
| Xanthan Gum | 0.6 | — |
| Hydroxypropyl Methylcellulose (HPMC) | — | 0.88 |
| Sodium Chloride | 0.47 | 0.52 |
| Polysorbate 80 | 0.05 | 0.05 |
| Monosodium Phosphate Dihydrate | 0.23 | 0.23 |
| Disodium Edetate Dihydrate | 0.01 | — |
| Benzododecinium Bromide | 0.012 | — |
| Benzalkonium Chloride | — | 0.01 |
| Sodium Hydroxide q.s. pH | 7.5 ± 0.1 | 7.5 ± 0.1 |
| Hydrochloric Acid q.s. pH | 7.5 ± 0.1 | 7.5 ± 0.1 |
| Purified Water q.s. % | 100 | 100 |

TABLE 5B
Effect of Xanthan Gum versus Hydroxypropyl Methylcellulose
(HPMC) on AL-34662 Assay (% of initial after correction for
weight loss) in formulations with Sodium Thiosulfate at 50° C.

FID

| | |
|---|---|
| 105215 | 105192 |

Lot Number

| | |
|---|---|
| 03-33366-1 | 03-33320-1 |

Critical Component

| | Xanthan Gum | HPMC |
|---|---|---|
| Age in Weeks | % Initial | % Initial |
| 0 | 100 | 100 |
| 4 | 99 | 97 |
| 8 | 98 | 96 |
| 12 | 97 | 94 |
| 16 | 96 | 92 |

TABLE 5C
Effect of Xanthan Gum versus Hydroxypropyl Methylcellulose (HPMC)
on the time it takes for the AL-34662 Assay to drop below 95%
and 90% of initial value in formulations with Sodium Thiosulfate
at 50° C.

FID

| | |
|---|---|
| 105215 | 105192 |

Lot Number

| | |
|---|---|
| 03-33366-1 | 03-33320-1 |

Critical Component

| | Xanthan Gum | HPMC |
|---|---|---|
| Age in weeks for AL- | More than 16 | Between 8 and |
| 34662 assay to drop | weeks | 12 weeks |
| below 95% label | | |
| Age in weeks for AL- | More than 16 | More than 16 |
| 34662 assay to drop | weeks | weeks |
| below 90% label | | |

### EXAMPLE 6

### Effect of Sodium Iodide, Sodium Sulfate, and Sodium Chloride Salts on the Stability of AL-34662 Formulations

Formulations of 1% AL-34662 with sodium iodide, sodium sulfate, and sodium chloride salts are listed in Table 6A. These formulations were prepared using the procedure described in Example 1. These formulations were packaged in standard 3 mL or 5 mL polyethylene DROP-TAINER® bottles and their stability was studied at 50° C. The results of the active compound (AL-34662) assay corrected for weight loss (which is usual for an aqueous product packaged in a semi-permeable container) are provided in Table 6B. The time it takes for the AL-34662 assay to drop below 95% of initial and 90% of initial is given in Table 6C. The results in Table 6C show that both sodium iodide and sodium sulfate prolong the stability of AL-34662 formulations compared to sodium chloride, i.e., the AL-34662 assay levels are above 90% of initial for a longer period of time.

TABLE 6A
Composition of formulations with Sodium Iodide, Sodium Sulfate, and
Sodium Chloride.

| | Formulation ID Number (FID) | | |
|---|---|---|---|
| | 104958 | 105193 | 105212 |
| | Lot Number | | |
| | 02-32843-1 | 03-33315-1 | 03-33353-1 |
| | Critical Component | | |
| | Sodium | Sodium | Sodium |
| | Iodide | Sulfate | Chloride |
| COMPONENT | % w/v | % w/v | % w/v |
| AL-34662 | 1 | 1 | 1 |
| Xanthan Gum | 0.6 | 0.6 | 0.6 |
| Sodium Iodide | 0.2 | — | — |
| Sodium Sulfate | — | 1.05 | — |
| Sodium Chloride | 0.42 | — | 0.5 |
| Polysorbate 80 | 0.05 | 0.05 | 0.05 |
| Monosodium Phosphate | 0.23 | 0.23 | 0.23 |
| Dihydrate | | | |
| Disodium Edetate Dihydrate | 0.01 | 0.01 | 0.01 |
| Benzododecinium Bromide | 0.012 | 0.012 | 0.012 |
| Sodium Hydroxide q.s. pH | 7.5 ± 0.1 | 7.5 ± 0.1 | 7.5 ± 0.1 |
| Hydrochloric Acid q.s. pH | 7.5 ± 0.1 | 7.5 ± 0.1 | 7.5 ± 0.1 |
| Purified Water q.s. % | 100 | 100 | 100 |

TABLE 6B
Effect of Sodium Iodide, Sodium Sulfate, and Sodium Chloride
on AL-34662 Assay (% of initial after correction for weight loss)
in formulations at 50° C.

| | FID | | |
|---|---|---|---|
| | 104958 | 105193 | 105212 |
| | Lot Number | | |
| | 02-32843-1 | 03-33315-1 | 03-33353-1 |
| | Critical Component | | |
| | Sodium | Sodium | Sodium |
| | Iodide | Sulfate | Chloride |
| Age in Weeks | % Initial | % Initial | % Initial |
| 0 | 100 | 100 | 100 |
| 4 | 96 | 100 | 100 |
| 8 | 94 | 92 | 84 |
| 12 | 94 | 75 | 73 |
| 16 | ND | 71 | 69 |
| 18 | 98 | ND | ND |

ND = Not Determined

TABLE 6C
Effect of Sodium Iodide, Sodium Sulfate, and Sodium Chloride
on the time it takes for the AL-34662 Assay to drop below
95% and 90% of initial value at 50° C.

| | FID | | |
|---|---|---|---|
| | 104958 | 105193 | 105212 |
| | Lot Number | | |
| | 02-32843-1 | 03-33315-1 | 03-33353-1 |
| | Critical Component | | |
| | Sodium | Sodium | Sodium |
| | Iodide | Sulfate | Chloride |
| Age in weeks for AL-34662 | Between 4 | Between 4 | Between 4 |
| assay to drop below 95% label | and 8 weeks | and 8 weeks | and 8 weeks |
| Age in weeks for AL-34662 | More than | Between 8 | Between 4 |
| assay to drop below 90% label | 18 weeks | and 12 | and 8 weeks |
| | | weeks | |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

We claim:

1. A stable ophthalmic solution comprising a compound with serotonergic (5-HT₂) receptor activity and one or more stabilizers selected from the group consisting of a thiosulfate salt, a thiosulfate hydrate, an iodide salt, an iodide hydrate, a sulfate salt, a sulfate hydrate, and xanthan gum.

2. The solution of claim 1, wherein the stabilizer is sodium thiosulfate pentahydrate.

3. The solution of claim 2, further comprising xanthan gum.

4. The solution of claim 1, wherein the stabilizer is sodium iodide.

5. The solution of claim 1, wherein the stabilizer is sodium sulfate.

6. The solution of claim 1, wherein the compound is an indazole derivative containing both a hydroxyl group on the six member ring and an aminoalkyl side chain on the five member ring.

7. The solution of claim 1, wherein the compound is selected from the group consisting of 1-(2-aminopropyl)-indazol-6-ol (AL-34662), 1-((S)-2-aminopropyl)-8,9-dihydropyrano[3,2-e]indole and (R)-1-((S)-2-aminopropyl)-1,7,8,9-tetrahydro-pyrano[2,3-g]indazol-8-ol.

8. The solution of claim 1, wherein the compound is 1-(2-aminopropyl)-indazol-6-ol (AL-34662).

9. The solution of claim 2, wherein the amount of serotonergic compound in the solution is from 0.01% to 5.0% and the amount of sodium thiosulfate pentahydrate is from 0.001% to 1.0%.

10. The solution of claim 3, wherein the amount of serotonergic compound in the solution is from 0.01% to 5.0%, the amount of sodium thiosulfate pentahydrate is from 0.001% to 1.0%, and wherein the amount of xanthan gum is from 0.1% to 1.0%.

11. The solution of claim 4, wherein the amount of serotonergic compound in the solution is from 0.01% to 5.0% and the amount of sodium iodide is from 0.01% to 2.0%.

12. The solution of claim 5, wherein the amount of serotonergic compound in the solution is from 0.01% to 5.0% and the amount of sodium sulfate is from 0.01% to 3.0%.

13. The solution of claim 9, wherein the amount of serotonergic compound in the solution is from 0.06% to 2.0% and the amount of sodium thiosulfate pentahydrate is from 0.01% to 0.2%.

14. The solution of claim 10, wherein the amount of serotonergic compound in the solution is from 0.06% to 2.0%, the amount of sodium thiosulfate pentahydrate is from 0.01% to 0.2%, and the amount of xanthan gum is from 0.3% to 0.8%.

15. The solution of claim 11, wherein the amount of serotonergic compound in the solution is from 0.06% to 2.0% and the amount of sodium iodide is from 0.1% to 0.5%.

16. The solution of claim 12, wherein the amount of serotonergic compound in the solution is from 0.06% to 2.0% and the amount of sodium sulfate is from 0.5% to 1.5%.

17. The solution of claim 13, wherein the amount of serotonergic compound in the solution is 1.0%, and the amount of sodium thiosulfate pentahydrate is 0.05%.

18. The solution of claim 14, wherein the amount of serotonergic compound in the solution is 1.0%, the amount of sodium thiosulfate pentahydrate is 0.05%, and the amount of xanthan gum is 0.6%.

19. The solution of claim 15, wherein the amount of serotonergic compound in the solution is 1.0%, and the amount of sodium iodide is 0.2%.

20. The solution of claim 16, wherein the amount of serotonergic compound in the solution is 1.0%, and the amount of sodium sulfate is 1.0%.

21. A method for treating glaucoma, said method comprising administering to a patient in need thereof a therapeutically effective amount of a solution of any one of claims 1-20.

22. A method for lowering intraocular pressure, said method comprising administering to a patient in need thereof a therapeutically effective amount of a solution of any one of claims 1-20.
